(19) United States
(12) Patent Application Publication
(10) Pub. No.: US 2012/0169995 A1
(43) Pub. Date: Jul. 5, 2012

(54) METHOD AND DEVICE FOR PRODUCING HIGH-QUALITY FUNDUS IMAGES

(75) Inventors: Thomas MOHR, Jena (DE); Björn ROßBERGER, Jena (DE)

(73) Assignee: CARL ZEISS MEDITEC AG, Jena (DE)

(21) Appl. No.: 13/232,703

(22) Filed: Sep. 14, 2011

(30) Foreign Application Priority Data
Sep. 14, 2010 (DE) 10 2010 045 364.1

(51) Int. Cl. A61B 3/14

(52) U.S. Cl. 351/206; 351/246

(57) ABSTRACT

To produce a color fundus image, the eye is illuminated with light pulses of defined wavelengths. Light reflected is recorded by a sensor and transmitted to a control unit. At least three monochromatic images at very short temporal intervals and a dark image of the fundus are recorded. After activation of a spectral-selective optical element, a color intensity distribution of the fundus is recorded by the sensor at white illumination. The monochromatic images are combined by the control unit to obtain a resulting color fundus image, wherein the color intensity distribution is used for the correction of color composition and the dark image is used for taking into account the noise of the sensor. The solution permits monitoring, documenting and/or diagnosing of the fundus and can also be executed with ophthalmological systems based on the principle of optical coherence and/or confocal imaging.

### FIELD OF THE INVENTION

The present invention relates to a method and a device for producing high-quality images of the fundus of an eye for later diagnosis through analysis of chromatic high-resolution images of the fundus.

### BACKGROUND

While, according to prior art, slit lamps or laser scanning ophthalmoscopes are, e.g., used for images of the entire eye structures, fundus cameras have become generally accepted for images of the fundus.

A fundus camera can map a precise image of the eyeground and thereby particularly depict the retina, the blood vessels, the optic nerve, and the choroid. It is used for diagnosis and for tracking the progress of diseases. Generally, the pupil of the patient must be dilated with medication when a fundus camera is used. It is known from prior art that no pupil reaction of the patient occurs in case of illumination of the eyeground by means of infrared light (invisible), therefore, a dilation of the pupil in a dark room occurs without the use of medication. This principle is utilized in so-called non-mydriatic fundus cameras. Once the pupil is sufficiently dilated, the eye is briefly illuminated with white (visible) light in order to record an image of the eyeground. Based on said principle, with a non-mydriatic fundus camera, infrared light is used to monitor and white light with shorter wavelength is used to record the result image. For 45° images, a typical non-mydriatic fundus camera provides a resolution of approximately 5 million pixels (5 Mpixels).

For producing color images, high-resolution CCD color sensors at broadband illumination, e.g., through xenon flashbulbs, are uses as a rule. CCD sensors (charge-coupled device) are light-sensitive and emit a signal proportional to the transmitted amount of light. They usually comprise a matrix with light-sensitive photodiodes which are called pixels. The larger the area of the pixels, the greater their light-sensitivity and the dynamic range of the CCD sensor, but the smaller their image resolution at the same sensor size. The light-sensitive elements of most CCD sensors are sensitive to the entire range of visible light and near-infrared light and, without additional measures, provide only grayscale values.

In order for CCD sensors to provide a signal proportional and wavelength-dependent to the transmitted amount of light, they exhibit a suitable arrangement of optical filter elements in front of the individual pixels. According to prior art, e.g., so-called Bayer patterns have become generally accepted, which comprise a symmetrical arrangement of one red, one blue as well as two green optical filters.

Furthermore, CMOS sensors are also used for producing fundus images. CMOS sensors are most notably less expensive but not as light-sensitive as CCD sensors and frequently exhibit distinct inhomogeneities in the sensitivity of the pixels.

The resolution of the color sensor is reduced by the Bayer pattern because 4 pixels of the Bayer pattern are combined to one color pixel for the RGB information of the color image. The typical image resolution of 5 to 8 million pixels in ophthalmological applications is therefore reduced to effectively 1.25 to 2 million pixels for a color sensor with Bayer pattern. This decrease in resolution of the color sensors additionally complicates the creation of high-resolution color images of the retina and therefore also an exact diagnosis.

According to prior art, the resolution can be increased, e.g., through interpolation techniques. However, experience has shown that an increase of the number of color pixels through combining 4 pixels of the Bayer pattern leads only theoretically to a higher resolution because in the field, no significant increase of the resolution was detected.

Another way to increase the resolution of color sensors would be the use of sensors with very high pixel numbers in the range of 10 to 15 million pixels. Since the observation pupil in the eye with a diameter of typically 1.0 to 1.5 mm limits the resolution through diffraction, this would also serve little purpose in ophthalmological applications.

For example, if a sensor with a detector surface of approximately 10×10 mm is used in ophthalmological applications, the result is a diffraction-limited image of approximately 5 μm on the sensor surface with a roughly estimated image of 1:1; therefore, resolutions greater than 2000×2000 pixels make no sense since the sensitivity significantly decreases with decreasing pixel dimensions.

According to prior art, systems and methods with an improved sensitivity for documenting an eye are known. Thereby, such systems utilize a narrow-band radiation source in conjunction with a highly sensitive black/white sensor with an image resolution in the range of 1.5 to 5 million pixels. With this system, three consecutive monochrome images are taken of the fundus and/or the anterior eye segments, wherein one (red, blue and/or green) color filter for each image is pivoted in front of the sensor. These three monochromatic single images can be combined to obtain a complete color image.

The solution described in DE 10 2005 034 332 A1 for monitoring, documenting and/or diagnosing of the fundus is also based on a multispectral, sequential illumination. Once again, monochromatic single images of a wavelength are averaged after a pixel-exact superimposition and melded with the averaged monochromatic images of other wavelengths to a resulting RGB image. In an advantageous embodiment, the resolution of the resulting image in this solution can be significantly increased, wherein n² images which are shifted against one another in the x-direction and independently in the y-direction by the n^{th} part of a pixel and which are entered in an image field of n^{th} size, i.e., nested into one another. The entire image field, which exhibits n times the size in each direction, is then unfolded in the Fourier space with a correction image and transformed back, resulting in an image with n-fold resolution.

A significant disadvantage of such sequential methods is the merging and/or superimposing of the monochromatic images to a color image through an algorithm. Thereto, according to the known prior art, features are extracted which are correspondingly present in all images. This requires a calculation-intensive image postprocessing which is error-prone to some extent because the features are frequently not present in an exactly reproducible fashion. This negatively affects the resolution as well as the image quality.

A further disadvantage of such a method is the relatively long exposure time of such a sequence of three monochromatic images. As a rule, color filters, which are moved by motor, wherein the filter movement lasts for approximately 25 to 50 ms, are used for filtering the radiation of the flashbulb. Depending on the resolution, the reading of an image from the electronic sensor takes approximately 20 to 70 ms. Therefore, approximately 400 ms must be expected for the recording of a complete RGB sequence with filter change and switch from the IR preview mode to the documentation mode at a resolution of 5 million pixels. Since the iris already closes after 120 to 180 ms in case of strong optical irritants, image sequences with a timeframe of approximately 400 ms are only possible in the mydriatic application. This poses a significant disadvantage. Furthermore, the required time for the calculation-intensive image postprocessing and the combining of the three monochromatic images, which, depending on the image content, can take from several seconds to several minutes, must be added to said timeframe of approximately 400 ms.

A multipixel color fundus camera is described in the yet to be published document DE 10 2009 043 749.5. In the method for producing high-quality images of the retina, parts of the retina, or even anterior eye segments, the eye is illuminated with infrared and monochromatic light of defined wavelengths, and the infrared and monochromatic light reflected from parts of the eye is recorded by at least one sensor and transmitted to a control unit for further processing, analysis, depiction and storage. Hereby, at least two image pairs, recorded simultaneously and/or immediately consecutively with infrared and monochromatic illumination of a defined wavelength are transmitted to the control unit. From the images of all image pairs recorded with infrared illumination, the control unit determines a possible pixel shift between the recorded image pairs and subsequently combines pixel-accurately the images of all image pairs recorded with monochromatic illumination with defined wavelengths to obtain a complete image.

The method described here provides a solution for producing high-resolution color images of the retina, parts of the retina, and even the anterior eye segments which is diffraction-limited in its resolution only by the eye pupil. The proposed method produces only nominal radiation exposures at the patient's eye and requires no calculation-intensive postprocessing. However, disadvantageously, disruptive influences of the measuring arrangement, e.g., the noise of the image acquisition sensor, still negatively affect the measurement results.

### SUMMARY OF THE INVENTION

The present invention addresses the problem of providing a solution for producing high-quality fundus images which eliminates the disadvantages of the solutions of prior art and with which fundus images with higher resolution and dynamic as well as improved image sharpness and decreased noise can be realized at a lower radiation exposure of the patient's eye.

The problem is solved, according to the invention, through the method for producing high-quality fundus images, wherein the eye is illuminated with light pulses of defined wavelengths, and wherein the light reflected from parts of the eye is recorded by at least one sensor and transmitted to a control unit for processing, analysis, depiction, and storage in such a way that at least three monochromatic images at defined wavelengths are recorded in very short temporal intervals and that subsequently a dark image of the fundus is recorded by at least one sensor and transmitted to the control unit; that a color intensity distribution of the fundus is recorded by the sensor after activation of a spectral-selective optical element and also transmitted to the control unit, and that the at least three monochromatic images are combined by the control unit to obtain a resulting color fundus image, wherein the color intensity distribution is used for the correction of the color composition and the dark image is used for taking into account the noise of the sensor.

According to the invention, the device for producing high-quality fundus images comprises a light source for illuminating an eye with light pulses of defined wavelengths, at least one sensor for recording the light reflected from parts of the eye, and a control unit for further processing, analysis, depiction, and storage of the images of the fundus transmitted by the sensor. Thereto, the sensor is designed in such way that it can record at least three monochromatic images at defined wavelengths in very short temporal intervals and subsequently record a dark image of the fundus and transmit them to the control unit. An activatable, spectral-selective optical element is provided in front of the sensor for recording a color intensity distribution of the fundus at white illumination. The control unit is capable of combining the at least three monochromatic images to obtain a resulting color fundus image, wherein the color intensity distribution is used for the correction of the color composition and the dark image is used for taking into account the noise of the sensor.

The solution, according to the invention, is provided for monitoring, documenting and/or diagnosing of the fundus of an eye, wherein the diagnosis takes place through analysis of chromatic high-resolution images of the retina or parts of the retina.

In principle, the suggested method can also be executed with ophthalmological systems which are based on the principle of optical coherence and/or confocal imaging, allowing for the combining of the scanned partial areas to obtain a complete image.

A device according to the invention includes the inventive aspects discussed below:

A device for producing high-quality fundus images, comprising:

a light source that illuminates the eye with light pulses of defined wavelengths;

at least one sensor that records light reflected from parts of the eye;

a control unit that further processes, analyses, depicts, and stores images of the fundus transmitted by the sensor;

wherein the sensor is designed such that it can record at least three monochromatic images at defined wavelengths at very short temporal intervals and record a dark image of the fundus and transmit the at least three monochromatic images and the dark image to the control unit;

the device further comprising an activatable, spectral-selective optical element that is provided in front of the sensor for recording a color intensity distribution of the fundus at white illumination;

wherein the control unit combines the at least three monochromatic images to obtain a resulting color fundus image and further wherein the color intensity distribution is used for the correction of the color composition and the dark image is used for taking into account the noise of the sensor.

In another example embodiment, the light source for illuminating the eye includes high-performance LED's or a xenon flash that produce light pulse of 200 to 500 μs duration and further comprising corresponding optical filters.

In another example embodiment, the activatable spectral-selective optical element comprises a pivotable optical grid, a prism, or a diffractive optical element.

In another example embodiment, the light source is designed such that light pulses with each one of the three wavelengths “red,” “green,” and “blue” are transmitted.

In another example embodiment, the light source is designed such that the light pulses with each one of the three wavelengths “red,” “green,” and “blue” are transmitted, in the order of the least irritation.

In another example embodiment, the light source is designed such that light pulses with each one of the four wavelengths “cyan,” “magenta,” “yellow,” and “green” are transmitted.

In another example embodiment, the light source is designed such that the light pulses with each one of the four wavelengths “cyan,” “magenta,” “yellow,” and “green” are transmitted in the order of the least irritation.

In another example embodiment, the at least one available sensor sectionally combines and transmits color intensity values to the control unit.

In another example embodiment, the filters exhibit filter characteristics which are adjusted to a color sensor, ensuring that the principal color composition corresponds to a typical color image.

Another example embodiment, further comprises a second sensor that records a color intensity distribution of the fundus at white illumination.

In another example embodiment, the second sensor is designed in the form of a corresponding number of single detectors, upon which the spectral-selective light portions are focused.

Another example embodiment, further comprises a mirror element 32 pivotable into position instead of a spectral-selective optical element if the second sensor is a color sensor.

In another example embodiment, the sensor is designed such that the monochromatic images and the color intensity distribution of the fundus are recorded within a timeframe of no more than 140 ms and transmitted to the control unit.

In another example embodiment, the sensor for the monochromatic images is highly level-controlled in order to ensure an optimal signal-to-noise ratio and a high dynamic resolution of the resulting color fundus image.

Another example embodiment, further comprises an additional sensor 34 that records the duration and intensity or the energy of the monochromatic illumination light during exposure and transmits the duration and intensity or the energy of the monochromatic illumination light with each image to the control unit.

In another example embodiment, the control unit determines the emitted optical energy during exposure from the duration and intensity or the energy of the monochromatic illumination light and assigns said energy to each image.

In another example embodiment, the control unit takes into account the emitted optical energy of the monochromatic image for the correction of the color composition of the resulting color fundus image.

In another example embodiment, the control unit is designed in such a way that the resolution of the color fundus image, resulting from the combined monochromatic images, can be increased through sub-pixel interpolation.

In another example embodiment, the device for producing high-quality fundus images, comprises a light source for illuminating an eye with light pulses of defined wavelengths, at least one sensor for recording the light reflected from parts of the eye, and a control unit for further processing, analysis, depiction, and storage of the images transmitted by the sensor, wherein the device is a fundus camera.

### BRIEF DESCRIPTION OF THE DRAWINGS

FIG. 1A is a flow chart according to an example embodiment of the invention;

FIG. 1B is a continuing flow chart according to an example embodiment of the invention;

FIG. 1C is a continuing flow chart according to an example embodiment of the invention;

FIG. 1D is a continuing flow chart according to an example embodiment of the invention; and

FIG. 2 is schematic depiction of a device according to the invention.

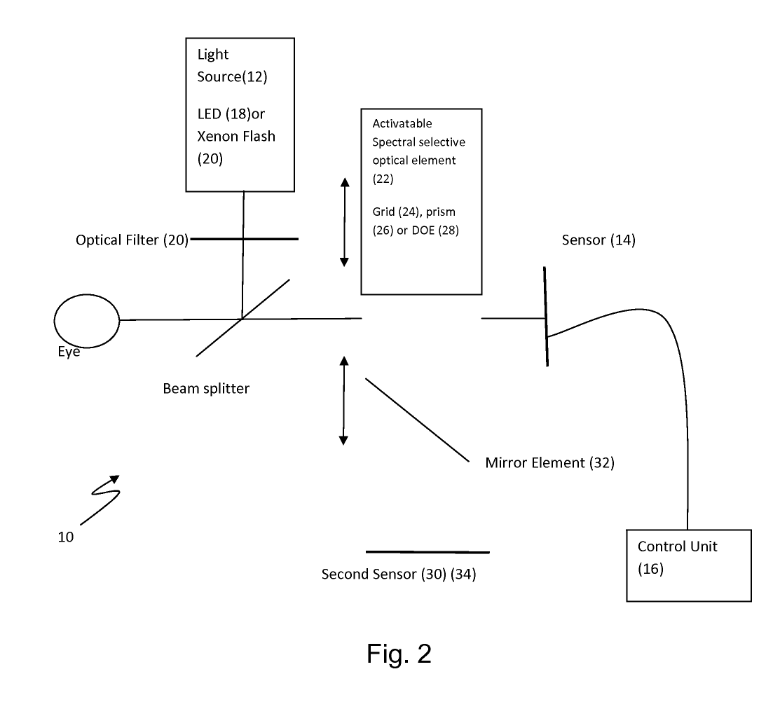

In the following, the invention is further described.

### DETAILED DESCRIPTION

With the method for producing high-quality fundus images, according to the invention, the eye is illuminated with light pulses of defined wavelengths, the light reflected from parts of the eye is recorded by at least one sensor and transmitted to a control unit for

further processing, analysis, depiction, and storage. Thereby, at least three monochromatic images at defined wavelengths are recorded in very short temporal intervals, and subsequently a dark image of the fundus is recorded by at least one sensor and transmitted to the control unit. After activation of a spectral-selective optical element, the sensor records a color intensity distribution of the fundus at white illumination, which is also transmitted to the control unit. The at least three monochromatic images are combined by the control unit to obtain a resulting color fundus image, wherein the color intensity distribution is used for the correction of the color composition and the dark image is used for taking into account the noise of the sensor.

While the dark image describes the noise of the sensor and is taken into account for the combining of the at least three monochromatic images, the color intensity distribution is used as guideline for the color combination of the resulting color fundus image.

Thereto, the color combination of the resulting color fundus image is adjusted to the color intensity distribution, significantly improving the dynamic of the image.

Therefore, producing high-quality fundus images takes place through combining a plurality of sequentially recorded images of the fundus, wherein different illumination conditions are applied. Specifically, five images are realized: Three monochromatic images, one dark image of the fundus, and finally a color intensity distribution.

Preferably, the spectral-selective optical element can be activated through pivoting in front of the sensor.

Hereby, high-performance LED's or a xenon flash with corresponding optical filters, the light pulses of which exhibit lengths of 200 to 500 μs, are used for illuminating the eye.

With such very short light pulses, unwanted eye movements can be largely avoided and a motion blur in the images does not occur. Therefore, a closing of the pupil of the patient's eye can be almost ruled out during the exposure time for the first four images.

In a first advantageous embodiment of the method, three monochromatic images are produced by the available high-resolution sensor. The monochromatic illumination is effected with each one of the wavelengths “red,” “green,” and “blue,” for example in the order of the least irritation for the eye. For example, an image in the red range with the least irritation should therefore be produced first. After an image is taken in the blue range with relatively little irritation, an image in the green range is recorded in which the eye is most sensitive and maximum irritation is caused. Therefore, the recording of the monochromatic images of the fundus takes place within a time frame of no more than 140 ms.

The color intensity distribution can be determined through producing a fundus image at white illumination. Thereto, a spectral-selective optical element is pivoted into the beam path by the sensor. The light reflected from the fundus is spectrally split by the spectral-selective optical element into at least three channels and focused on three different sections of the sensor. Therefore, the sensor is capable of reading color intensity values by sector and transmitting them to the control unit. Analogous to the images of the fundus recorded with monochromatic illumination, the light is spectral-selectively split into the wavelengths “red,” “green,” and “blue.”

Optical grids as well as diffractive optical elements or prisms can be used as spectral-selective optical element.

For example, a dark image of the fundus, i.e., an image without illumination, is subsequently recorded and transmitted to the control unit. The three monochromatic images are combined by the control unit to obtain a resulting color fundus image, wherein the color intensity distribution is used for the correction of the color composition and the dark image is used for taking into account the noise of the sensor.

In a second example embodiment of the method, four monochromatic images are produced by the available high-resolution sensor. As a result, the color space as well as the resolution (at sub-pixel interpolation) can be further improved. The CMYG model with the colors “cyan,” “magenta,” “yellow,” and “green” would be a possible color model for four colors. Advantageously, the four images are also taken in the order of the least irritation.

Once again, the color intensity distribution is produced through a fundus image at white illumination, wherein the light reflected from the fundus is spectrally split by the spectral-selective optical element into the four channels and focused on four different sections of the sensor. Analogous to the first embodiment, the light is spectral-selectively split into the four wavelengths “cyan,” “magenta,” “yellow,” and “green.”

For example, a dark image of the fundus, i.e., an image without illumination, is subsequently once again recorded and transmitted to the control unit. The four monochromatic images are combined by the control unit to obtain a resulting color fundus image, wherein the color intensity distribution is used for the correction of the color composition and the dark image is used for taking into account the noise of the sensor.

In a third example embodiment of the method, occurring longitudinal chromatic aberrations can be offset through a wavelength-specific focus shift, wherein the focus is individually adjusted for every wavelength. As a result, the focus can be individually adjusted for all monochromatic images, and therefore all images exhibit a consistently very good image sharpness. The focus shift is preferably applied to mechanically operated objectives.

A fourth example embodiment provides that the filter characteristics of the employed filters are adjusted to a color sensor, ensuring that the principal color composition corresponds to a typical color image. Therefore, the resulting color fundus image, produced by the control unit from the monochromatic images, exhibits a comparable color quality.

In a further embodiment of the method, more than only one sensor is used for mapping the light reflected from parts of the eye. Specifically, the color intensity distribution of the fundus is recorded by a second sensor at white illumination and transmitted to the control unit.

Thereby, a corresponding number of single detectors or a color sensor can be used as second sensor.

If a corresponding number of single detectors is used, the light reflected from parts of the eye must also be appropriately split by a spectral-selective optical element and focused on the single detectors. Accordingly, a spectral-selective optical element must be pivoted in front of the single detectors for the recording of the color intensity distribution of the fundus at white illumination. In the simplest case, photodiodes are hereby used as single detectors.

By contrast, if a color sensor is used, it suffices to pivot a mirror element instead of a spectral-selective optical element as second sensor. Said element maps the light reflected from parts of the eye onto the entire color sensor which realizes a color image of the fundus. Preferably, the applied color sensor is a CCD-type sensor.

Regardless of the number and type of the applied sensors, the monochromatic images are combined to obtain a resulting color fundus image by the control unit, wherein the color intensity distribution is used for the correction of the color composition and the dark image is used for taking into account the noise of the sensor.

However, if a color sensor is used, the color composition is realized through a simple color image of the fundus at white illumination instead of a color intensity distribution. Accordingly, the resulting color fundus image is adjusted to the color histogram of said color image. This significantly improves the dynamic of the resulting color fundus image.

In a further advantageous embodiment of the method, the monochromatic images are recorded with a level-controlled sensor in order to ensure an optimal signal-to-noise ratio and a high dynamic resolution of the resulting color fundus image. Thereby, the level of control of the individual images should be at approximately 70%. The result is an optimal signal-to-noise ratio for every monochromatic image. By means of the known color composition of the color sensor, the control unit subsequently determines a chromatic fundus image with an optimal signal-to-noise ratio and very high dynamic resolution.

In a further example embodiment, the duration and the intensity of the monochromatic illumination light is recorded during exposure, a measure for the emitted optical energy is determined and assigned to each image and transmitted to the control unit. Alternatively, the energy of the respective monochromatic images can be measured. This allows for a control of the intensity and/or energy, preset correspondingly for the monochromatic illumination and for an individual determination of type-related or age-related fluctuations of the radiation sources in order to further improve the reproducibility with regard to brightness and color composition. Type-related or age-related intensity fluctuations of the radiation source can, e.g., make the comparison of fundus images more difficult or even impossible.

Furthermore, the values of the (actually) emitted optical energy can additionally be taken into account by the control unit during the combining of the monochromatic images to obtain a resulting color fundus image, particularly during the correction of the color composition. As a result, the signal-to-noise ratio and the dynamic resolution of the resulting color fundus image can, in addition to the color fidelity, be further improved.

Hereby, it is particularly advantageous if the values of the emitted optical energy are utilized for the analysis of the illumination as well as for producing a resulting color fundus image.

In a further advantageous embodiment of the method, the resolution of the resulting color fundus image, combined by the control unit from the monochromatic images, is further increased through sub-pixel interpolation.

The use of a fundus camera for executing the method, according to the invention, is to be deemed a further embodiment of the method.

For example, the fundus camera “VISUCAM” from Carl Zeiss Meditec AG is applicable and must for this purpose be equipped with a xenon flash with appropriate filters and the necessary software for the control unit because, as a rule, an electronic sensor which operates in the monochromatic as well as the color mode is already present.

The device 10 for producing high-quality fundus images, according to the invention, comprises a light source 12 for illuminating the eye with light pulses of defined wavelengths, at least one sensor 14 for recording the light reflected from parts of the eye, and a control unit 16 for further processing, analysis, depiction, and storage of the images of the fundus transmitted by the sensor 14. Thereby, the sensor 14 is designed in such way that it can record at least three monochromatic images at defined wavelengths in very short temporal intervals and subsequently record a dark image of the fundus and transmit them to the control unit 16. An activatable, spectral-selective optical element 22 is provided in front of the sensor for recording a color intensity distribution of the fundus at white illumination. Furthermore, the control unit 16 is capable of combining the at least three monochromatic images to obtain a resulting color fundus image, wherein the color intensity distribution is used for the correction of the color composition and the dark image is used for taking into account the noise of the sensor.

While the dark image describes the noise of the sensor and is taken into account for the combining of the at least three monochromatic images to obtain a resulting fundus image, the color intensity distribution is used as guideline for the color combination of the resulting color fundus image. Thereto, the color combination of the resulting color fundus image is adjusted to the color intensity distribution, significantly improving the dynamic of the image.

Therefore, producing high-quality fundus images takes place through combining a plurality of sequentially recorded images of the fundus, wherein different illumination conditions are applied. Specifically, five images are realized: Three monochromatic images, one dark image of the fundus, and finally a color intensity distribution.

Preferably, the activatable, spectral-selective optical element is a pivotable optical grid 24, a prism 26, or a diffractive optical element 28.

Hereby, high-performance LED's 16 or a xenon flash 18 with light pulse lengths of 200 to 500 μs and which exhibits corresponding optical filters, are preferably used as a light source for illuminating the eye.

With such very short light pulses, unwanted eye movements can be largely avoided and a motion blur in the images does not occur. Therefore, a closing of the pupil of the patient's eye can be almost ruled out during the exposure time for the first four images.

The light source is designed in such a way that light pulses can be transmitted with each one of the singular wavelengths “red,” “green,” and “blue,” preferably in the order of the least irritation of the eye, as well as a “complete” spectrum. For the monochromatic illumination with each one of the wavelengths “red,” “green,” and “blue,” the corresponding filters are pivoted into and out of the beam path. Preferably, an image in the red range with the least irritation should therefore be produced first. After an image is taken in the blue range with relatively little irritation, an image in the green range is recorded in which the eye is most sensitive and maximum irritation is caused.

Furthermore, the sensor is designed in such a way that the monochromatic images as well as the image of the color intensity distribution of the fundus can be recorded within a timeframe of no more than 140 ms and transmitted to the control unit.

With such a very short time interval, unwanted eye movements can be largely avoided and elaborate determinations and corrections of the shifts between the single images can be foregone. Therefore, a closing of the pupil of the patient's eye as wells as its movement can be almost ruled out during the exposure time for the first four images.

Advantageously, for determining the color intensity distribution, the sensor records a fundus image at white illumination. Thereto, a spectral-selective optical element is pivoted into the beam path by the sensor. The light reflected from the fundus is spectrally split by the spectral-selective optical element into at least three channels and focused on three different sections of the sensor. Therefore, the sensor is capable of reading color intensity values by sector and transmitting them to the control unit. Analogous to the images of the fundus recorded with monochromatic illumination, the light is spectral-selectively split into the wavelengths “red,” “green,” and “blue.”

For example, the sensor subsequently also records a dark image of the fundus, i.e., an image without illumination. The control unit is designed in such a way that the three monochromatic images are combined to obtain a resulting color fundus image, wherein the color intensity distribution is used for the correction of the color composition and the dark image is used for taking into account the noise of the sensor.

In a second advantageous embodiment of the device, the light source is designed in such a way that light pulses are transmitted with each one of four wavelengths, preferably in the order of the least irritation. Accordingly, the sensor is capable of recording four monochromatic images as well as the image of the color intensity distribution of the fundus within a timeframe of no more than 140 ms and of transmitting them to the control unit.

As a result, the color space as well as the resolution (at sub-pixel interpolation) can be further improved. The CMYG model with the colors “cyan,” “magenta,” “yellow,” and “green” would be a possible color model for four colors.

For determining the color intensity distribution, the sensor once again records a fundus image at white illumination. Thereto, a spectral-selective optical element is pivoted into the beam path by the sensor. The light reflected from the fundus is spectrally split by the spectral-selective optical element into the four channels and focused on four different sections of the sensor. Analogous to the images of the fundus recorded with monochromatic illumination, the light is spectral-selectively split into the four wavelengths “cyan,” “magenta,” “yellow,” and “green.”

Once again, the sensor also records a dark image of the fundus. The control unit combines the four monochromatic images to obtain a resulting color fundus image, wherein the color intensity distribution is used for the correction of the color composition and the dark image is used for taking into account the noise of the sensor.

In a third advantageous embodiment of the device, the filters exhibit a filter characteristic which is adjusted to a color sensor, ensuring that the principal color composition corresponds to a typical color image.

A fourth advantageous embodiment provides that the filter characteristics of the employed filters are adjusted to a color sensor, ensuring that the principal color composition corresponds to a typical color image. Therefore, the resulting color fundus image, produced by the control unit from the monochromatic images, exhibits a comparable color quality.

A further embodiment of the device provides that the sensor for the monochromatic images is highly level-controlled in order to ensure an optimal signal-to-noise ratio and a high dynamic resolution of the resulting color fundus image. The level of control of the individual images should be at approximately 70%. As a result, an optimal signal-to-noise ratio can be achieved for every monochromatic image. By means of the known color composition of the color sensor, the control unit subsequently determines a chromatic fundus image with an optimal signal-to-noise ratio and very high dynamic resolution.

In a further advantageous embodiment of the device, according to the invention, more than only one sensor is provided for mapping the light reflected from parts of the eye. Specifically, a second sensor 30 is provided for mapping the color intensity distribution of the fundus at white illumination.

Hereby, the second sensor can be a corresponding number of single detectors or a color sensor.

If the second sensor consists of a corresponding number of single detectors, the light reflected from parts of the eye must also be split by a spectral-selective optical element. Once again, the spectral-selective optical element is designed as a pivotable element. In the simplest case, photodiodes are hereby used as single detectors.

By contrast, if the second sensor is a color sensor, a mirror element 32 instead of a spectral-selective optical element is required. Said element maps the light reflected from parts of the eye onto the entire color sensor which realizes a color image of the fundus. Preferably, the applied color sensor is a CCD-type sensor.

Hereby, the color composition of the resulting color fundus image is realized through a simple color image of the fundus at white illumination instead of a color intensity distribution. Accordingly, the resulting color fundus image is adjusted to the color histogram of said color image. This significantly improves the dynamic of the resulting color fundus image.

A further embodiment of the device, according to the invention, provides an additional sensor in the form of a photodiode for mapping a part of the monochromatic illumination light. As a rule, 2-3% of monochromatic illumination light is sufficient. The photodiode records the duration and the intensity of the monochromatic illumination light during exposure, a measure for the emitted optical energy is determined, assigned to each image and transmitted to the control unit. This allows for a control of the intensity and/or energy, preset correspondingly for the monochromatic illumination and for an individual determination of type-related or age-related fluctuations of the radiation sources in order to further improve the reproducibility with regard to brightness and color composition. Type-related or age-related intensity fluctuations of the radiation source can, e.g., make the comparison of fundus images more difficult or even impossible.

Furthermore, the control unit can be designed in such a way that the values of the (actually) emitted optical energy can additionally be taken into account by the control unit during the combining of the monochromatic images to obtain a resulting color fundus image, particularly during the correction of the color composition. As a result, the signal-to-noise ratio and the dynamic resolution of the resulting color fundus image can, in addition to the color fidelity, be further improved.

Thereby, it is particularly advantageous if the values of the emitted optical energy are utilized for the analysis of the illumination as well as for producing a resulting color fundus image.

Regardless of the number and type of the applied sensors, the control unit is designed in such a way that the monochromatic images are combined to obtain a resulting color fundus image, wherein the color intensity distribution is used for the correction of the color composition and the dark image is used for taking into account the noise of the sensor.

A further embodiment of the device, according to the invention, provides for the control unit to be capable of increasing the resolution of the color fundus image, which results from the monochromatic images, through sub-pixel interpolation.

For depicting the images of the fundus, which were recorded by the sensor and transmitted to the control unit, and the resulting color fundus images, determined by the control unit, the control unit exhibits a display.

In a final embodiment, the device for producing high-quality fundus images, according to the invention, is a fundus camera.

For example, the fundus camera “VISUCAM” from Carl Zeiss Meditec AG is applicable and must for this purpose be equipped with a xenon flashbulb with appropriate filters and necessary software for the control unit because, as a rule, an electronic sensor which operates in the monochromatic as well as the color mode is already present.

The method, according to the invention, and the corresponding device provide a solution which eliminates the disadvantages of the solutions of prior art and with which fundus images with higher resolution and dynamic as well as improved image sharpness and decreased noise can be realized with lower radiation exposure of the patient's eye.

The development of motion blurs can be avoided and improved image sharpness therefore achieved with the use of high-performance LED's or a xenon flashbulb with very short pulses of 200 to 300 μs.

The already achieved high resolution of the resulting color fundus image can be further increased through sub-pixel interpolation.

Furthermore, the dynamic of the resulting fundus image is increased through adjusting its color composition to the color histogram of the color image.

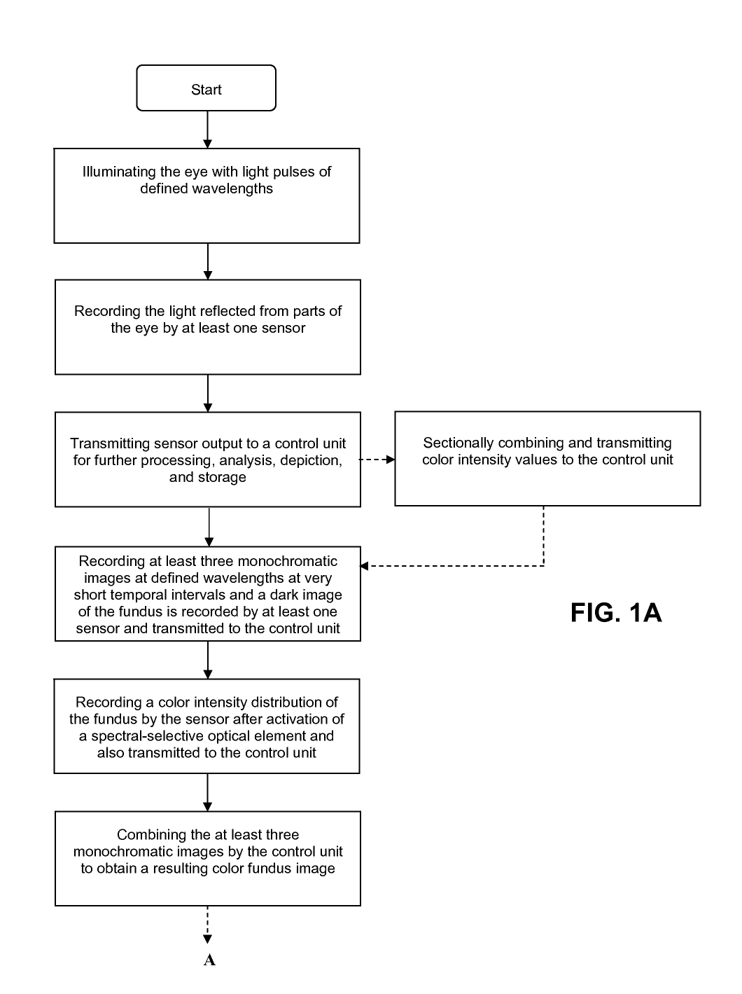

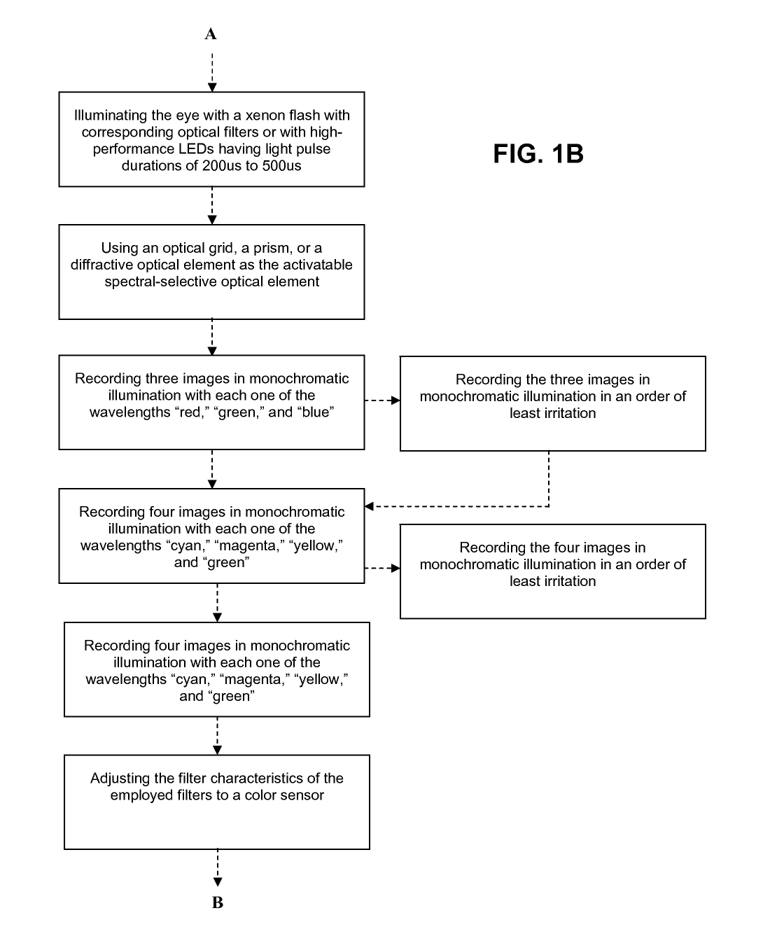

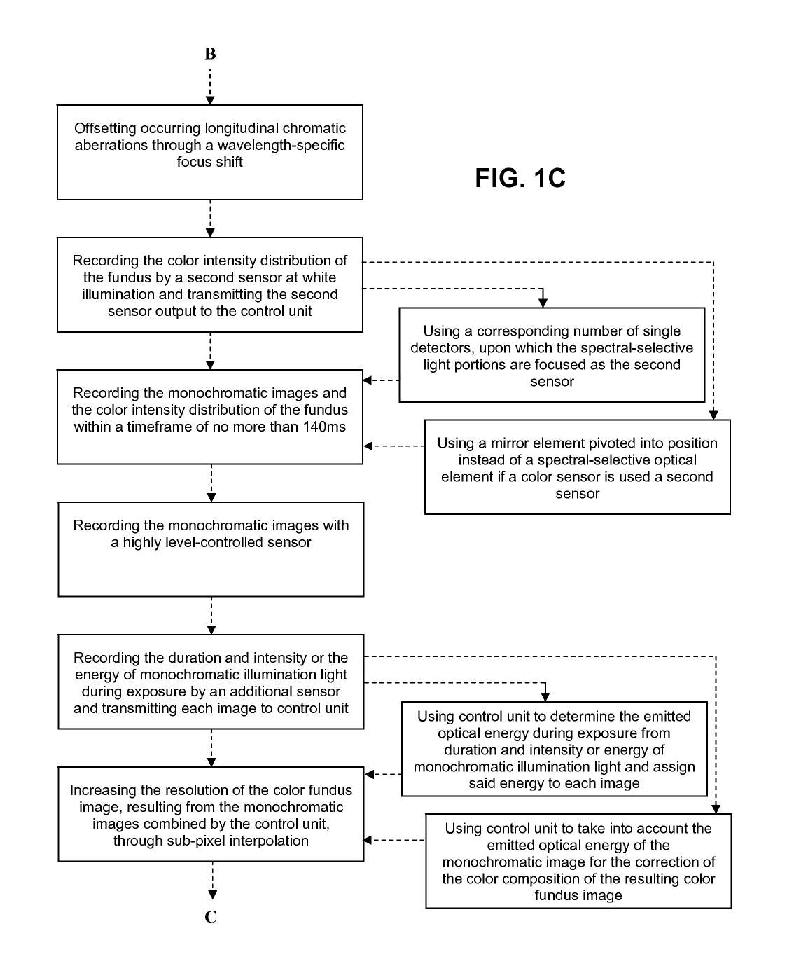

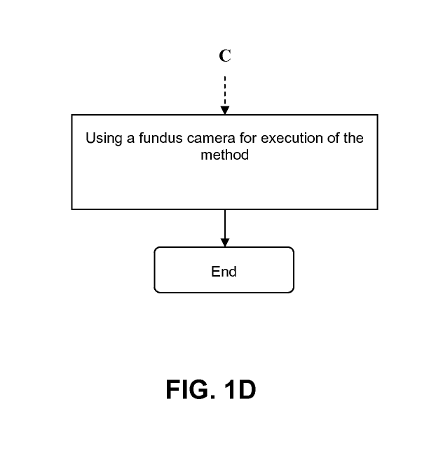

The flow charts of FIGS. 1A-1D describe example embodiments of a method according to the invention. The flowcharts should not be considered limiting and the invention includes methods as recited in the claims that may include only portions of the subject matter depicted in FIGS. 1A-1D.

1. A Method for producing high-quality fundus images, comprising:
illuminating the eye with light pulses of defined wavelengths;
recording light reflected from parts of the eye with at least one sensor;
transmitting sensor output to a control unit for further processing, analysis, depiction, and storage;
recording at least three monochromatic images at defined wavelengths at very short temporal intervals and a dark image of the fundus with the at least one sensor and transmitting the three monochromatic images and the dark image to the control unit;
recording a color intensity distribution of the fundus by the at least one sensor after activation of a spectral-selective optical element and transmitting the color intensity distribution to the control unit; and
combining the at least three monochromatic images with the control unit to obtain a resulting color fundus image, wherein the color intensity distribution is used for the correction of the color composition and the dark image is used for taking into account the noise of the sensor.

2. The method, according to claim 1, further comprising illuminating the eye with a xenon flash with corresponding optical filters or illuminating the eye with high-performance LED's, light pulses of which have durations of 200 to 500 μs.

3. The method, according to claim 1, further comprising using an optical grid, a prism, or a diffractive optical element as the activatable spectral-selective optical element.

4. The method, according to claim 1, further comprising recording three images in monochromatic illumination with each one of the wavelengths “red,” “green,” and “blue”.

5. The method, according to claim 4, further comprising recording the three images in monochromatic illumination in an order of least irritation.

6. The method, according to claim 1, further comprising recording four images in monochromatic illumination with each one of the wavelengths “cyan,” “magenta,” “yellow,” and “green”.

7. The method, according to claim 6, further comprising recording the four images in monochromatic illumination in an order of least irritation.

8. The method, according to claim 1, wherein the at least one available sensor sectionally combines and transmits color intensity values to the control unit.

9. The method, according to claim 1, further comprising adjusting the filter characteristics of the employed filters to a color sensor, ensuring that the principal color composition corresponds to a typical color image.

10. The method, according to claim 1, further comprising offsetting occurring longitudinal chromatic aberrations through a wavelength-specific focus shift, wherein the focus is individually adjusted for every wavelength.

11. The method, according to claim 1, further comprising recording the color intensity distribution of the fundus by a second sensor at white illumination and transmitting the second sensor output to the control unit.

12. The method, according to claim 11, further comprising using a corresponding number of single detectors, upon which the spectral-selective light portions are focused as the second sensor.

13. The method, according to claim 11, further comprising using a mirror element pivoted into position instead of a spectral-selective optical element if a color sensor is used as second sensor.

14. The method, according to claim 1, further comprising recording the monochromatic images and the color intensity distribution of the fundus within a timeframe of no more than 140 ms.

15. The method, according to claim 1, further comprising recording the monochromatic images with a highly level-controlled sensor in order to ensure an optimal signal-to-noise ratio and a high dynamic resolution of the resulting color fundus image.

16. The method, according to claim 1, further comprising recording the duration and intensity or the energy of the monochromatic illumination light during exposure by an additional sensor and transmitting each image to the control unit.

17. The method, according to claim 16, further comprising using the control unit to determine the emitted optical energy during exposure from the duration and intensity or the energy of the monochromatic illumination light and to assign said energy to each image.

18. The method, according to claim 16, further comprising using the control unit to take into account the emitted optical energy of the monochromatic image for the correction of the color composition of the resulting color fundus image.

19. The method, according to claim 1, further comprising, increasing the resolution of the color fundus image, resulting from the monochromatic images combined by the control unit, through sub-pixel interpolation.

20. The method, according to claim 1, further comprising, using a fundus camera for execution of the method.
